(19) United States
(12) Patent Application Publication
(10) Pub. No.: US 2020/0245855 A1
(43) Pub. Date: Aug. 6, 2020

(54) MEDICAL OBSERVATION APPARATUS AND MEDICAL OBSERVATION SYSTEM

(71) Applicant: Sony Olympus Medical Solutions Inc., Tokyo (JP)

(72) Inventors: Takahiro TAMURA, Tokyo (JP); Takamasa MIKAMI, Kanagawa (JP); Tatsuya NAKANISHI, Tokyo (JP)

(73) Assignee: Sony Olympus Medical Solutions Inc., Tokyo (JP)

(21) Appl. No.: 16/699,133

(22) Filed: Nov. 29, 2019

(30) Foreign Application Priority Data
Feb. 6, 2019 (JP) 2019-020136

(51) Int. Cl. A61B 1/12; A61B 1/00; A61B 1/05

(52) U.S. Cl. CPC A61B 1/128; A61B 1/00119; A61B 1/05; A61B 1/00124; A61B 1/00128; A61B 1/00114

(57) ABSTRACT

A medical observation apparatus includes: a grip configured to be connected to an insertion portion inserted into a subject, the grip being gripped by a user; a transmission cable extending from the grip; a connector provided at an end portion of the transmission cable and configured to be detachably connected to a controller outside the medical observation apparatus; a heat generator provided inside the insertion portion or the grip, and configured to generate heat when driven; a signal transmission path included in the transmission cable, and configured to perform signal transmission between the controller and the grip; and a cooling channel configured to distribute a cooling fluid thermally connected to the heat generating portion, the cooling channel being an annular channel configured to circulate the cooling fluid within the medical observation apparatus.

The present application claims priority to and incorporates by reference the entire contents of Japanese Patent Application No. 2019-020136 filed in Japan on Feb. 6, 2019.

### BACKGROUND

The present disclosure relates to a medical observation apparatus and a medical observation system.

There is a known medical observation system including: an endoscope to be inserted into a subject to capture light (subject image) reflected from the subject; and a control device to which the endoscope is detachably connected and configured to cause a display device to display an endoscope image corresponding to the subject image captured by the endoscope (refer to JP 2006-664 A, for example).

In the medical observation system described in JP 2006-664 A, the endoscope includes: an imaging unit that images a captured subject image; and a cooling unit that cools the imaging unit. The cooling unit includes a refrigerant recirculation pipe wound around an outer peripheral surface of the imaging unit, and cools the imaging unit by distributing a cooling fluid along a cooling channel in the refrigerant recirculation pipe.

### SUMMARY

Incidentally, in order to forcibly distribute the cooling fluid along the cooling channel in the medical observation system described in JP 2006-664 A, there would be a need to provide a circulator such as a pump. An example of a configuration for forcibly distributing the cooling fluid along the cooling channel (hereinafter referred to as a forced cooling configuration) is a configuration including a cooling channel provided across an endoscope and a control device, and including a circulator within the control device. However, in a case where the forced cooling configuration is employed, the cooling channel is structurally divided in accordance with attachment/detachment of the endoscope to/from the control device. Accordingly, there is a need to select a structure that causes no problem even when the cooling fluid leaks to the outside, or have a structure that may prevent leakage of the fluid to the outside using a valve, or the like. In addition, in a case where the valve fails, this configuration would also have a problem of leakage of the cooling fluid to the outside from the attachment/detachment portion.

According to one aspect of the present disclosure, there is provided a medical observation apparatus including: a grip configured to be connected to an insertion portion inserted into a subject, the grip being gripped by a user; a transmission cable extending from the grip; a connector provided at an end portion of the transmission cable and configured to be detachably connected to a controller outside the medical observation apparatus; a heat generator provided inside the insertion portion or the grip, and configured to generate heat when driven; a signal transmission path included in the transmission cable, and configured to perform signal transmission between the controller and the grip; and a cooling channel configured to distribute a cooling fluid thermally connected to the heat generating portion, the cooling channel being an annular channel configured to circulate the cooling fluid within the medical observation apparatus.

### BRIEF DESCRIPTION OF THE DRAWINGS

FIG. 1 is a view illustrating a schematic configuration of a medical observation system according to a first embodiment;

FIG. 2 is a view illustrating a configuration of a medical observation apparatus;

FIG. 3 is a view illustrating a configuration inside a camera head;

FIG. 4 is a view illustrating a configuration inside a connector according to a second embodiment; and

FIG. 5 is a view illustrating a modification of the first and second embodiments.

### DETAILED DESCRIPTION

Hereinafter, modes for carrying out the present disclosure (hereinafter referred to as embodiments) will be described with reference to the drawings. The present disclosure is not limited to the embodiments described below. In the description of the drawings, the identical reference numerals will be used to denote identical portions.

### First Embodiment

Schematic Configuration of Medical Observation System

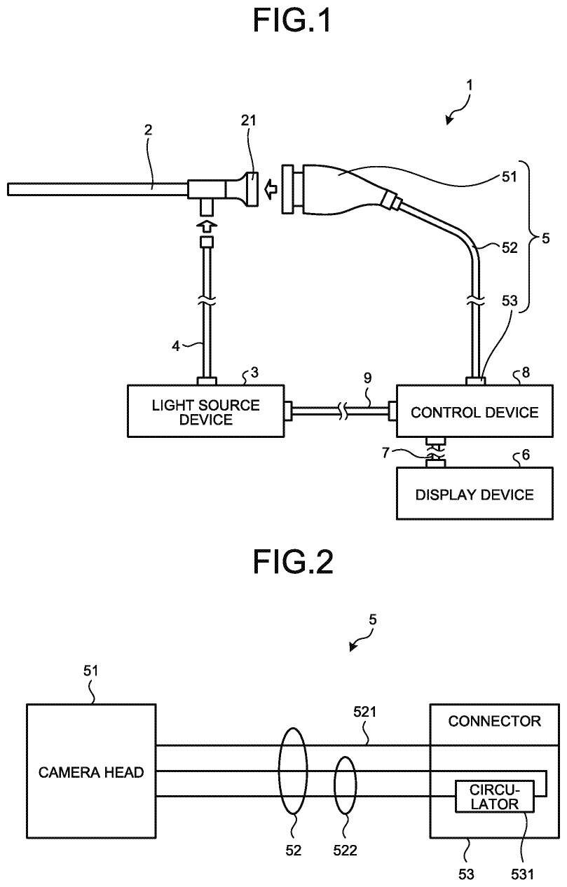

FIG. 1 is a view illustrating a schematic configuration of a medical observation system 1 according to a first embodiment.

The medical observation system 1 is a system that is used in the medical field and observes a subject such as inside of a living body. As illustrated in FIG. 1, the medical observation system 1 includes an insertion portion 2, a light source device 3, a light guide 4, a medical observation apparatus 5, a display device 6, a first transmission cable 7, a control device 8, and a second transmission cable 9.

In the first embodiment, the insertion portion 2 is implemented by a rigid endoscope. That is, the insertion portion 2 has an elongated shape that is entirely rigid, or partially rigid with a partially flexible portion, so as to be inserted into a living body. The insertion portion 2 includes an optical system having one or more lenses and configured to collect light (subject image) from the living body.

The light source device 3 is connected to one end portion of the light guide 4, and supplies light for illuminating the inside of the living body to one end portion of the light guide 4 under the control of the control device 8.

In the present embodiment, the light source device 3 is separated from the control device 8. However, the configuration is not limited thereto, and it is allowable to employ a configuration in which the light source device 3 is provided inside the control device 8.

The light guide 4 has one end portion detachably connected to the light source device 3 and the other end portion detachably connected to the insertion portion 2. The light guide 4 transmits the light supplied from the light source device 3 from one end portion to the other end portion and supplies the light to the insertion portion 2. The light supplied to the insertion portion 2 is emitted from a distal end portion of the insertion portion 2 and directed into the living body. The light (subject image) directed into the living body is collected by the optical system in the insertion portion 2.

The medical observation apparatus 5 is detachably connected with a proximal end portion (an eyepiece 21 (FIG. 1)) of the insertion portion 2, while detachably connected to the control device 8. The medical observation apparatus 5 captures the subject image collected by the insertion portion 2, and outputs an image signal (RAW signal) obtained by the imaging to the control device 8. In the first embodiment, the image signal is an image signal of 4K resolution or more.

A detailed configuration of the medical observation apparatus 5 will be described below.

The display device 6 is implemented by a display using liquid crystal, organic Electro Luminescence (EL), or the like, and displays an image based on a video signal from the control device 8 under the control of the control device 8.

The first transmission cable 7 has one end portion detachably connected to the display device 6 and has the other end portion detachably connected to the control device 8. The first transmission cable 7 transmits the video signal processed by the control device 8 to the display device 6.

The control device 8 is implemented by a central processing unit (CPU), a Field-Programmable Gate Array (FPGA), or the like, and comprehensively controls operation of the light source device 3, the medical observation apparatus 5, and the display device 6.

Specifically, the control device 8 performs predetermined processing on the image signal acquired from the medical observation apparatus 5 and thereby generates a video signal, and then outputs the video signal to the display device 6 via the first transmission cable 7. In response to this, the display device 6 displays an image based on the video signal. The control device 8 outputs a control signal or the like to the light source device 3 and the medical observation apparatus 5.

The second transmission cable 9 has one end portion detachably connected to the light source device 3 and the other end portion detachably connected to the control device 8. The second transmission cable 9 transmits a control signal or the like from the control device 8 to the light source device 3.

Configuration of Medical Observation Apparatus

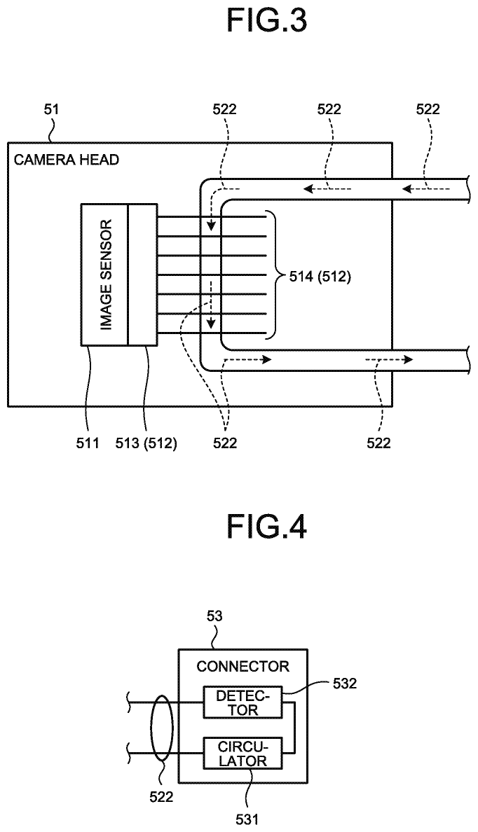

FIG. 2 is a view illustrating a configuration of the medical observation apparatus 5. FIG. 3 is a view illustrating a configuration inside a camera head 51.

As illustrated in FIGS. 1 to 3, the medical observation apparatus 5 includes the camera head 51, a third transmission cable 52 (FIGS. 1 and 2), and a connector 53 (FIGS. 1 and 2).

The camera head 51 is a portion to which the eyepiece 21 of the insertion portion 2 is detachably connected and that is gripped by a user such as an operator, and thus, corresponds to a grip portion. The camera head 51 includes a lens unit (not illustrated), an image sensor 511 (FIG. 3), a signal processing unit (not illustrated), and a communication unit (not illustrated).

The lens unit includes one or more lenses, and forms a subject image collected by the insertion portion 2 on an imaging surface of the image sensor 511.

The image sensor 511 is implemented by a Charge Coupled Device (CCD), Complementary Metal Oxide Semiconductor (CMOS) or the like that receives the subject image formed by the lens unit and converts the image into an electrical signal (analog signal).

The signal processing unit performs signal processing on the electrical signal output from the image sensor 511 and transmits the processed signal to the communication unit.

The communication unit transmits the image signal output from the signal processing unit to the control device 8 via the third transmission cable 52. For example, the communication unit includes a high-speed serial interface that performs image signal communication with the control device 8 via the third transmission cable 52 at a transmission rate of 1 Gbps or more.

Here, the image sensor 511 corresponds to a heat generating portion. As illustrated in FIG. 3, a heat sink 512 is thermally connected directly to the image sensor 511.

As illustrated in FIG. 3, the heat sink 512 includes a heat receiving block 513 and a plurality of fins 514.

The heat receiving block 513 is a plate formed of a metal material or the like having high thermal conductivity, and is thermally connected directly to the image sensor 511. The heat receiving block 513 receives the heat generated in the image sensor 511.

The plurality of fins 514 each has a thin flat plate shape formed of a metal material or the like having high thermal conductivity. The plurality of fins 514 each protrudes from the heat receiving block 513 in a posture that is parallel to each other. That is, the heat received by the heat receiving block 513 from the image sensor 511 is transmitted to each of the plurality of fins 514.

The connector 53 is a portion that is mechanically connected to the control device 8, and is provided at the distal end portion of the third transmission cable 52.

The third transmission cable 52 is a portion extending from the camera head 51 and corresponds to the transmission cable. The third transmission cable 52 is detachably connected to the control device 8 via the connector 53. As illustrated in FIG. 2, the third transmission cable 52 includes a signal transmission path 521 and a cooling channel 522.

The signal transmission path 521 is a signal transmission path that performs signal transmission between the control device 8 and the camera head 51. More specifically, the signal transmission path 521 transmits the image signal output from the camera head 51 to the control device 8, and transmits each of the control signal, synchronization signal, clock, power, or the like output from the control device 8 to the camera head 51. For convenience of explanation, FIG. 2 illustrates one signal transmission path alone as the signal transmission path 521. However, a plurality of signal transmission paths is provided in an actual situation.

Note that the image signal transmitted from the camera head 51 to the control device 8 via the signal transmission path 521 may be transmitted in an optical signal or an electrical signal. The similar applies to transmission of the control signal, the synchronization signal, and the clock from the control device 8 to the camera head 51 via the signal transmission path 521.

The cooling channel 522 is a channel that is formed by a pipe or the like formed of a metal material or the like having high thermal conductivity so as to distribute the cooling fluid. In the first embodiment, as illustrated in FIG. 2, the cooling channel 522 is implemented as an annular channel circulating the inside of the connector 53 to the third transmission cable 52 to the inside of the camera head 51, back into the connector 53 through the third transmission cable 52. That is, the cooling channel 522 is implemented as an annular channel that circulates the cooling fluid within the medical observation apparatus 5.

In addition, the cooling channel 522 passes through the front and back of the plurality of fins 514 in the camera head 51, as illustrated in FIG. 3. That is, the cooling fluid is thermally connected to the image sensor 511 via the plurality of fins 514.

In the connector 53, a circulator 531 is provided on the cooling channel 522 as illustrated in FIG. 2. The circulator 531 is electrically connected to the control device 8 via the connector 53, and circulates the cooling fluid along the cooling channel 522 so as to distribute the cooling fluid at a specific flow rate under the control of the control device 8. An example of the circulator 531 is a pump. The cooling fluid may be a gas or a liquid.

The cooling fluid circulated by the circulator 531 receives heat through the plurality of fins 514, and dissipates the received heat during distribution inside the third transmission cable 52. Then, the cooling fluid is distributed again toward the plurality of fins 514. As described above, the cooling fluid circulates along the cooling channel 522, whereby the image sensor 511 is cooled.

According to the first embodiment described above, the following effects are obtained.

In the medical observation apparatus 5 according to the first embodiment, the cooling channel 522 that distributes the cooling fluid for cooling the image sensor 511 is implemented as an annular channel that circulates the cooling fluid in the medical observation apparatus 5. That is, the cooling channel 522 is not provided across the medical observation apparatus 5 and other devices such as the control device 8, and thus, would not be divided in accordance with the attachment/detachment of the medical observation apparatus 5 to/from the other devices.

Therefore, according to the medical observation apparatus 5 of the first embodiment, the cooling fluid may be kept in the cooling channel 522, making it possible to reduce the leakage of the cooling fluid to the outside.

In the medical observation apparatus 5 of the first embodiment, the cooling channel 522 is implemented as an annular channel circulating the inside of the connector 53 to the third transmission cable 52 to the inside of the camera head 51, back to the connector 53 through the third transmission cable 52.

With this configuration, it is possible to dissipate the heat of the cooling fluid during distribution through the third transmission cable 52 extending relatively long. That is, it is possible to efficiently cool the image sensor 511 without necessity to separately provide a heat dissipation device that dissipates the heat of the cooling fluid.

In addition, the medical observation apparatus 5 according to the first embodiment includes, inside the connector 53, the circulator 531 that is located on the cooling channel 522 and circulates the cooling fluid.

For this reason, it is possible to provide, inside the connector 53, a connection portion for connecting the pipe or the like forming the cooling channel 522 and the circulator 531. This configuration makes it possible to reduce the leakage of the cooling fluid to the outside of the medical observation apparatus 5.

### Second Embodiment

Next, a second embodiment will be described.

In the following description, identical reference numerals are given to the components similar to those in the first embodiment described above, and detailed description thereof will be omitted or simplified.

FIG. 4 is a view illustrating a configuration inside the connector 53 according to the second embodiment. For convenience of explanation, FIG. 4 omits illustration of the signal transmission path 521.

The second embodiment is different from the first embodiment described above only in that a detector 532 is provided in the connector 53.

The detector 532 is electrically connected to the control device 8 via the connector 53, and detects leakage of the cooling fluid to the outside the cooling channel 522 under the control of the control device 8. In the second embodiment, the detector 532 is provided on the cooling channel 522 and is implemented as a flow meter that measures the flow rate of the cooling fluid, for example. The detector 532 outputs a signal corresponding to the measured flow rate, and the signal is processed in the connector 53 or in the control device 8. In a case where the flow rate measured by the detector 532 is below a specific flow rate, it is determined that there is a leakage of the cooling fluid to the outside of the cooling channel 522. In that case, processing such as stopping the operation of the circulator 531 would be performed. In addition, the control device 8 causes the display device 6 to display information indicating that the use of the medical observation system 1 is to be stopped, for example.

According to the second embodiment described above, the following effects are obtained in addition to the effects similar to the case of the first embodiment described above.

The connector 53 according to the second embodiment includes the detector 532 that detects leakage of the cooling fluid to the outside of the cooling channel 522.

Therefore, when the detector 532 detects leakage of the cooling fluid leaks to the outside of the cooling channel 522, it is possible to stop the operation of the circulator 531. This configuration makes it possible to further reduce the leakage of the cooling fluid to the outside of the medical observation apparatus 5.

### Other Embodiments

While the above is description of the modes for carrying out the present disclosure, the present disclosure should not be limited by only the first and second embodiments described above.

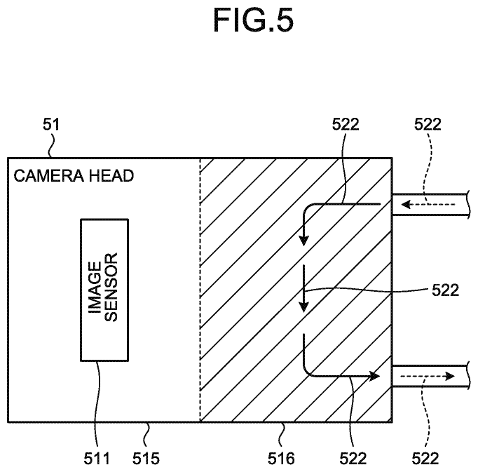

FIG. 5 is a view illustrating a modification of the present first and second embodiments. Specifically, FIG. 5 is a view that corresponds to FIG. 3.

In the first and second embodiments described above, the cooling fluid is thermally connected to the image sensor 511 via the heat sink 512. However, the present disclosure is not limited to this, and the cooling fluid may be thermally connected to the image sensor 511 in a manner as illustrated in FIG. 5.

Specifically, the camera head 51 includes an airtight part 515 and a filling part 516 as illustrated in FIG. 5. In FIG. 5, for convenience of explanation, the boundary between the airtight part 515 and the filling part 516 is represented by a broken line.

The airtight part 515 is a case that is formed of a metal material or the like having high thermal conductivity and that accommodates the image sensor 511 in an airtight manner, and is thermally connected to the image sensor 511.

The filling part 516 is formed of a metal material having high thermal conductivity and is thermally connected directly to the airtight part 515. The filling part 516 is filled with a cooling fluid. In FIG. 5, for convenience of explanation, the cooling fluid is represented by diagonal lines. The filling part 516 constitutes a part of the cooling channel 522. That is, the cooling fluid is thermally connected to the image sensor 511 through the filling part 516 and the airtight part 515.

Although the first and second embodiments described above employ the forced cooling configuration in which the cooling fluid is forcibly distributed along the cooling channel 522 in the circulator 531, the present disclosure is not limited to this, and the cooling fluid may be naturally distributed along the cooling channel 522 without providing the circulator 531. Furthermore, it is also allowable to employ a configuration in which the circulator 531 is provided outside the medical observation apparatus 5. At this time, for example, it is possible to employ a magnetic fluid as the cooling fluid, and employ a configuration in which a magnetic field is applied to the magnetic fluid from outside the medical observation apparatus 5 or from within the cable as the circulator 531. The magnetic fluid is distributed along the cooling channel 522 in response to the application of the magnetic field.

Although the above-described first and second embodiments employ the image sensor 511 the heat generating portion, the present disclosure is not limited to this. A configuration other than the image sensor 511 provided in the camera head 51 or a configuration provided in the insertion portion 2 may be employed as the heat generating portion.

In Embodiment 2 described above, the detector 532 is provided in the connector 53, but the present disclosure is not limited to this. The detector may be provided at other positions as long as the position is on the cooling channel 522 or close to the cooling channel 522.

In the first and second embodiments described above, the medical observation apparatus is mounted on the medical observation system 1 using a rigid endoscope. However, the present disclosure is not limited thereto, and the medical observation apparatus may be mounted on the medical observation system using a flexible endoscope.

In the first and second embodiments described above, the cooling fluid is circulated along the cooling channel 522 so that the cooling fluid is distributed at a specific flow rate under the control of the control device 8. The present disclosure, however, is not limited to this circulation. For example, a control circuit may be provided in the camera head 51 or the connector 53, and the cooling fluid may be circulated along the cooling channel 522 so that the cooling fluid is distributed at a specific flow rate under the control of the control circuit.

In the first and second embodiments described above, it is also allowable to have a configuration for efficiently transmitting the heat generated in the image sensor 511 to the airtight part 515 by using a material having high thermal conductivity such as metal for an adapter for securing the image sensor 511 in the camera head 51. For example, it is allowable to use, as an adapter, a metal such as aluminum disposed around the lens in the lens unit and around the image sensor 511.

According to the medical observation apparatus and the medical observation system, it is possible to reduce leakage of the cooling fluid that cools the heat generating portion to the outside.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A medical observation apparatus comprising:
a grip configured to be connected to an insertion portion inserted into a subject, the grip being gripped by a user;
a transmission cable extending from the grip;
a connector provided at an end portion of the transmission cable and configured to be detachably connected to a controller outside the medical observation apparatus;
a heat generator provided inside the insertion portion or the grip, and configured to generate heat when driven;
a signal transmission path included in the transmission cable, and configured to perform signal transmission between the controller and the grip; and
a cooling channel configured to distribute a cooling fluid thermally connected to the heat generating portion, the cooling channel being an annular channel configured to circulate the cooling fluid within the medical observation apparatus.

2. The medical observation apparatus according to claim 1, wherein
the cooling channel passes through the connector, and
the connector includes a circulator located on the cooling channel and configured to circulate the cooling fluid.

3. The medical observation apparatus according to claim 1, wherein
the grip is a camera head to which the insertion portion is detachably connected, and
the heat generator is an image sensor provided in the camera head and configured to image a subject image taken by the insertion portion.

4. The medical observation apparatus according to claim 1, further comprising a detector configured to detect leakage of the cooling fluid to an outside of the cooling channel.

5. A medical observation system comprising:
the medical observation apparatus according to claim 1; and
a controller to which the medical observation apparatus is detachably connected.
